(19) United States
(12) Patent Application Publication
(10) Pub. No.: US 2016/0361505 A1
(43) Pub. Date: Dec. 15, 2016

(54) An Injection Apparatus

(71) Applicant: NOVO NORDISK A/S, Bagsværd (DK)

(72) Inventors: Henrik Bengtsson, Taastrup (DK); Vera Pinto Glenting, Copenhagen (DK); Emil Gram Spork, Copenhagen N (DK); Elisabeth Diget, Taastrup (DK); Lene Bay, Broenshoej (DK)

(21) Appl. No.: 15/120,828

(22) PCT Filed: Jan. 30, 2015
(86) PCT No.: PCT/EP2015/051964

§ 371 Date: Aug. 23, 2016

(30) Foreign Application Priority Data
Feb. 24, 2014 (EP) 14156344.5

(51) Int. Cl. A61M 5/42; A61M 5/31; A61M 5/32; A61M 5/24

(52) U.S. Cl. CPC A61M 5/422; A61M 5/2459; A61M 5/31; A61M 5/3202; A61M 2005/3121

(57) ABSTRACT

The present invention relates to an injection apparatus in which a cartridge has a distal end sealed by a pierceable flexible septum abutting a pressure element. Further a shielded needle cannula penetrates the distal septum. This pressure element is axially movable whenever the shield is axially moved such that the volume of cartridge decreases. A motion transforming means transforms axial movement of the shield into axial movement of the pressure element via a rotational movement of the motion transforming means.

### THE TECHNICAL FIELD OF THE INVENTION

The invention relates to an injection apparatus having a shielded needle cannula. The invention especially relates to such injection device having a mechanism which automatic expels drops of the liquid drug contained in the injection device when the needle shield is moved axially during injection.

### DESCRIPTION OF RELATED ART

Shielded needle assemblies are widely known. WO 2003/045480 discloses a safety needle assembly in which the axially movable shield locks after one injection such that the needle assembly cannot be reused.

WO 2008/077706 discloses a similar construction wherein the telescopic shield is prevented from axial movement when the needle assembly is removed from the injection device. However, every time the needle assembly is mounted onto the injection device the shield unlocks.

### DESCRIPTION OF THE INVENTION

It is an object of the present invention to provide an injection device in which the tip of the needle cannula automatically is made wet prior to or during penetrating the skin of the user.

The invention is defined in the attached claim 1 followed by a number of embodiments. The individual claims are explained in details in the following.

Accordingly, in one aspect of the present invention, the injection apparatus comprises:
A cartridge having a distal end which sealed by a pierceable flexible septum and an opposite proximal end sealed by a movable plunger. The wall of the cartridge together with the flexible septum and the movable plunger defines an interior space containing a liquid drug,
A needle cannula defining a hollow longitudinal lumen through which the liquid drug can be expelled. The needle cannula is attached to a hub means, and comprises a distal part and a proximal part. The proximal part penetrates the flexible septum such that liquid drug can flow from the interior of the cartridge and through the lumen of the needle cannula upon movement of the movable plunger. The distal end preferably penetrates the skin of a user during injection
A telescopic shield which move telescopic in relation to the hub means and which shield is urged in a distal direction by a spring.
A motion transforming means which engages the telescopic shield and transforms an axial movement of the telescopic shield to a rotational movement of the motion transforming means, and
An axially movable pressure element engaging the motion transforming means and which pressure element is configured to perform an axial movement relatively to the cartridge upon rotational movement of the motion transforming means.

Further, axial movement of the telescopic shield in a proximal direction rotates the motion transforming means which operates the pressure element to move axially in a proximal direction, and wherein the pressure element abuts the flexible septum which is forced proximally upon axial movement of the pressure element such that the volume of the interior of the cartridge decreases.

In this way a gearing is established between the axial movement of the shield and the axial movement of the pressure element. The result being that a drop of liquid drug is pressed out through the lumen of the needle cannula upon axial movement of the shield such that the tip of the needle cannula is wet during injection.

When the tip of the needle cannula is wet during injection, the friction as the needle penetrates the skin of the user is minimized thus making the injection less pain full. Further due to the preservatives comprised in the liquid drug, bacteria transmission is minimized as the lumen of the needle cannula is flushed as the telescopic shield moves axially.

The motion transforming means is preferably formed as one unitary element but could be constructed from a number of parts working together to transform axial movement to rotational movement. In the same manor, the hub means can be one unitary unit or it can be assembled from a number of parts which when together form the hub means.

In a preferred embodiment, the tip of the needle cannula is maintained submerged in a cleaning solvent between injections. This cleaning solvent is preferably stored in a reservoir carried by the shield. In a preferred embodiment, the cleaning solvent is either the same preservative as present in the liquid drug or alternatively the cleaning reservoir is filled with the exact same drug as contained in the cartridge. This reservoir preferably has a proximal surface and a distal surface made from a pierceable septum material. During injection, the tip of the needle cannula penetrates through the distal septum such that the tip projects distally to perform an injection. During the penetration of the distal septum it is an advantage that the tip of the needle cannula is made increasingly more wet since this cleans the tip as it cuts through the distal septum.

In one embodiment, the hub means form part of an injection device such as a pre-filled injection device i.e. an injection device which is not to be re-filled by the user.

In another embodiment, the hub means form part of a needle assembly which is attachable to an injection device which can be either pre-filled or durable.

The telescopic shield preferably comprises an internal track for guiding the motion transforming means. This track preferably has a helical track part for introducing rotation to the motion transforming means as it move axially. The motion transforming means comprises an axial extending hollow part surrounding the needle cannula and a perpendicular bar having two opposite ends. At least one end of the perpendicular bar engages the helical part of the internal track of the telescopic shield. Further, the motion transforming means is axially secured such that the motion transforming means can only rotate around its own centre axis (X) upon axial movement of the telescopic shield.

As the shield is moved axially, without rotation, upon injection, the engagement between the shield and the motion transforming means forces the motion transforming means to rotate. In an alternative embodiment, the track can be provided in the motion transforming means and the engaging protrusion can be provided in the shield. Thus the important feature being that at least a part of the motion transforming means rotate as the shield move axially.

The proximal end of the hollow part of the motion transforming means preferably has a wave-shaped surface which engages with a similar wave-shaped distal end surface of a pressure element.

As the pressure element is axial guided preferably in the hub means, the pressure element is forced to move axially as the motion transforming means is rotated. The gearing ratio between the axial movement of the shield and the axial movement of the pressure element is thus a result of the pitch of the helical part of the track and the shape of the waves.

The axial and proximal movement of the pressure element is transferred to a proximal depressive movement of the flexible septum of the cartridge since the pressure element abuts the flexible septum.

The result being that the volume of the interior of the cartridge is decreased which again presses liquid drug out through the lumen of the connected needle cannula. The amount of liquid drug thus expelled is equal to the volume the interior of the cartridge is decreased.

### Definitions:

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and connected to a hub to form a complete injection needle also often referred to as a “needle assembly”. A needle cannula could however also be made from a polymeric material or a glass material. The hub also carries the connecting means for connecting the needle assembly to an injection apparatus and is usually moulded from a suitable thermoplastic material. The “connection means” could as examples be a luer coupling, a bayonet coupling, a threaded connection or any combination thereof e.g. a combination as described in EP 1,536,854.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 shows a cross sectional view of the injection apparatus prior to performing an injection.

FIG. 2 shows a cross sectional view of the injection apparatus commencing the injection.

FIG. 3 shows a cross sectional view of the application during injection.

FIG. 4 shows a perspective cut-open view of the telescopic shield.

FIG. 5 shows a perspective view of the motion transforming means.

FIG. 6 shows a close-up sectional view of the interface between the motion transforming means and the pressure element in the non-dosing position of FIG. 1.

FIG. 7 shows a close-up sectional view of the interface between the motion transforming means and the pressure element in the dosing position of FIG. 3.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

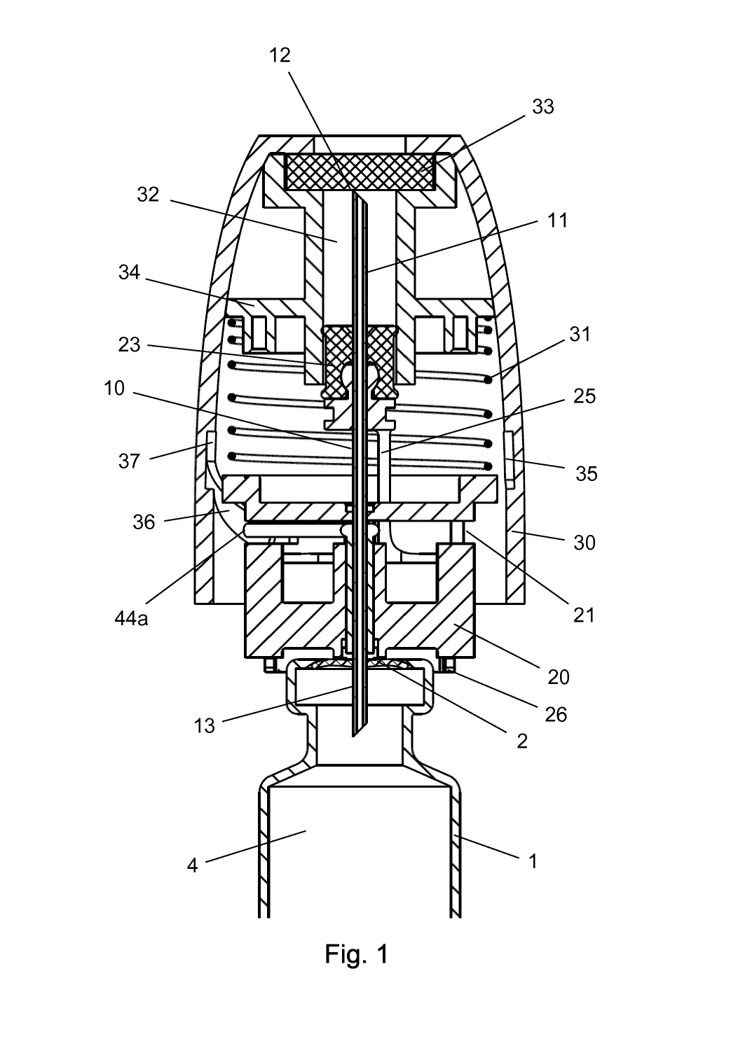

In that context it may be convenient to define that the term “distal end” in the appended figures is meant to refer to the end of the needle cannula supposed to be inserted into the skin of the user whereas the term “proximal end” is meant to refer to the opposite end pointing away from the user and usually inserted into the interior of the cartridge as depicted in FIG. 1.

The injection apparatus as depicted in FIGS. 1 to 3 comprises a cartridge 1 having a distal flexible septum 2 and a proximal movable plunger 3. The wall of the cartridge 1, the distal septum 2 and the movable plunger 3 together defines an interior 4 which contains a liquid drug.

The distal flexible septum 2 is penetrated by a needle cannula 10. The needle cannula 10 has a distal part 11 with a tip 12 and a proximal part 13 and defines a lumen 14. The proximal part 13 is penetrated through the distal septum 2 of the cartridge 1 such that axial movement of the movable plunger 3 in a distal direction presses the liquid drug out through the lumen 14 of the needle cannula 10.

The needle cannula 10 is secured in a hub 20. The hub 20 can as in the depicted embodiment be permanent attached to an injection device such that the needle assembly and the injection device form one single unit which is disposed after use. Alternatively, in a different and non-shown embodiment, the hub 20 and thus the needle assembly can be releasable connected to the injection device e.g. by a thread or a bayonet connection such that the needle assembly can be discarded without discarding the entire injection device.

Sliding on the outside of the hub 20 is the telescopic shield 30. The shield 30 is urged in a distal direction by a spring 31 encompassed between the hub 20 and the shield 30. The shield 30 further carries a reservoir 32 for keeping the tip 12 of the needle cannula 10 clean between subsequent injections.

Such cleaning reservoir 32 for cleaning the tip 12 of the needle cannula 10 between injections is further described in International patent application No.: PCT/EP2014/071746. Distally this reservoir 32 has a distal septum 33 carried by the shield 30 and proximally the reservoir 32 is bordered by a proximal septum 23 which is carried by an auxiliary element 25 sliding in the hub 20. Since the reservoir 32 is filled with a cleaning liquid, which in a preferred embodiment is identical to the liquid drug contained in the interior 4 of the cartridge 1, the volume of the reservoir 32 remains constant. The auxiliary element 25 thus moves axially together with the reservoir 32 and the telescopic shield 30. The auxiliary element 25 is preferably provided with stop protrusions 26 such that the auxiliary element 25 cannot slide distally out of its engagement with the hub 20.

As depicted in FIG. 1-3 the distal septum 33 is secured inside the shield 30 by an insert 34 which also form the base for the spring 31.

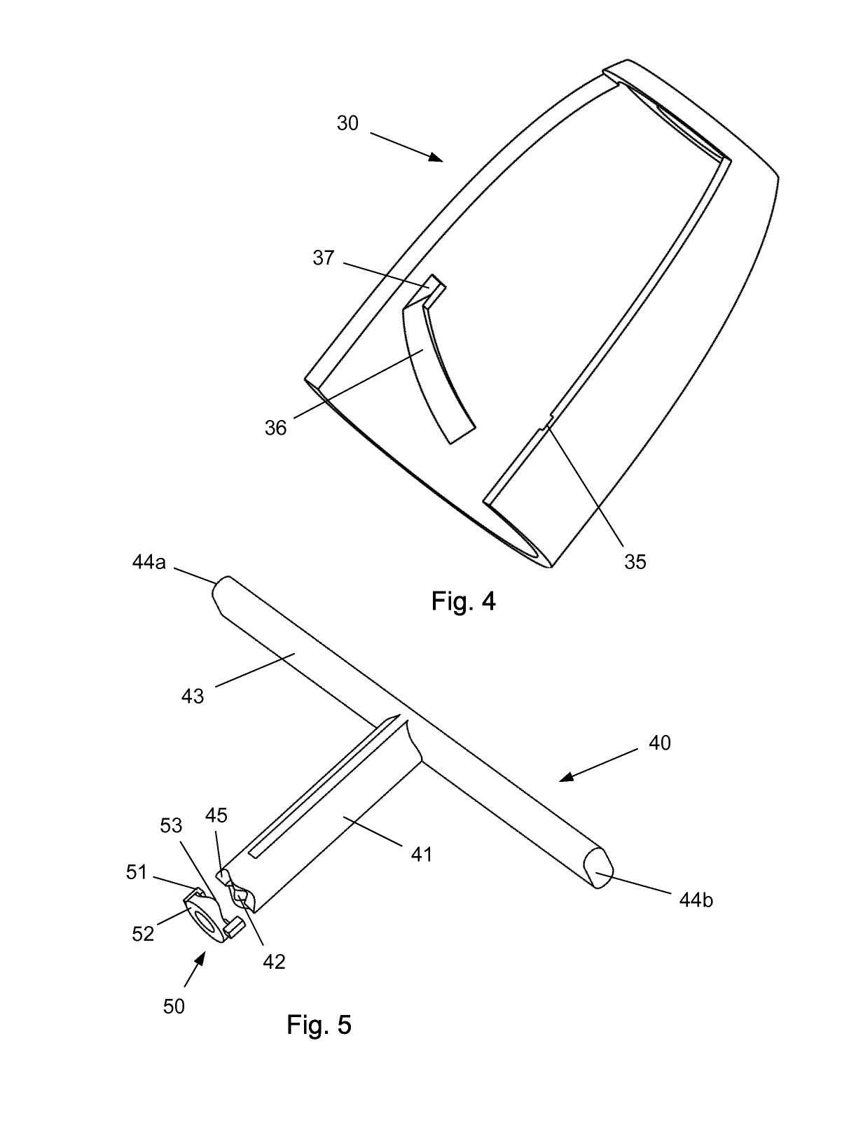

The telescopic shield 30 is internally provided with a track 35 which has a helical part 36 and an axial part 37 as depicted in FIG. 4.

Guided in the hub 20 and surrounding the needle cannula 10 is the motion transforming element 40. This motion transforming element 40, which is depicted in perspective in FIG. 5 comprises an axial hollow part 41 which has a through-going opening 42 which surrounds the needle cannula 10. Proximally the axial hollow part 41 has a perpendicular bar 43 having two ends 44a-b. The perpendicular bar 43 is axially locked in the hub 20 such that the motion transforming element 40 can only rotate without any axial movement. Further, the axial hollow part 41 has a proximal end 45 provided with a wave-shape, the function of which will be explained later.

The hub 20 is preferably provided with a cut-out 21 in which the perpendicular bar 43 of the motion transforming element 40 is axially locked but able to rotate a predetermined angle.

At least one end 44a of the perpendicular bar 43 engages the internal track 35 in the telescopic shield 30 as disclosed in FIGS. 1 to 3. Alternatively both the end 44a-b can engage the track 35 in which case the shield 30 would have two such tracks 35.

FIG. 1 discloses the position of the telescopic shield 30 prior to performing an injection. The tip 12 of the needle cannula 10 is maintained submerged in the cleaning agent contained in the reservoir 32 and at least one end 44a of the perpendicular bar 43 is positioned in the bottom (proximal end) of the track 35.

The track (or tracks) 35 have a helical part 36 and an axial part 37 as will be explained.

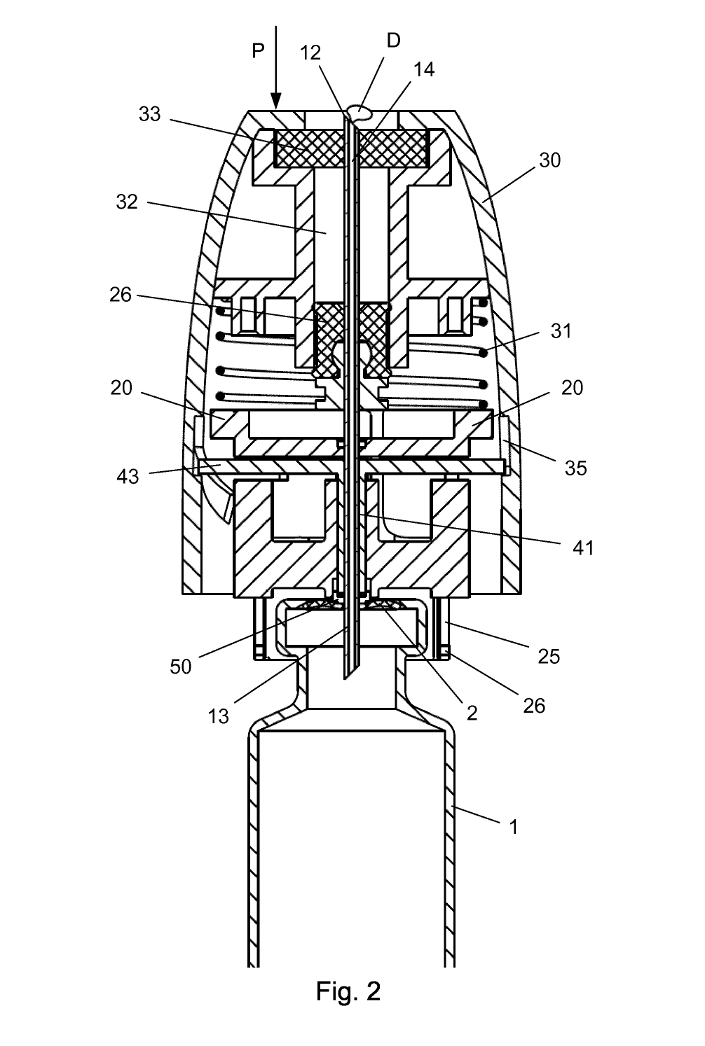

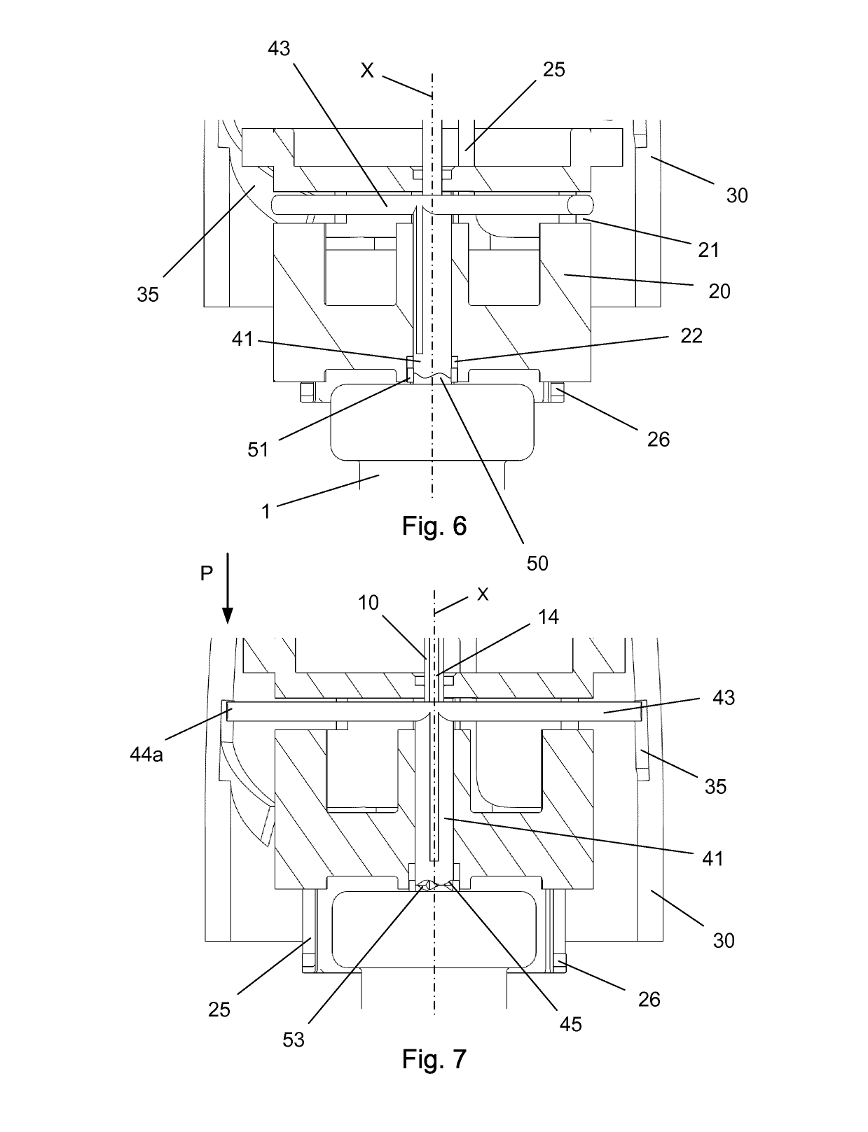

As an injection is performed, the user presses the shield 30 against the skin which pushes the shield 30 in a proximal direction as indicated by the arrow “P” in FIG. 2. The motion transforming element 40 is thereby forced to perform a rotational movement around its own centre axis “X” (FIG. 6-7) due the helical part 36 of the track 35 of the telescopic shield 30.

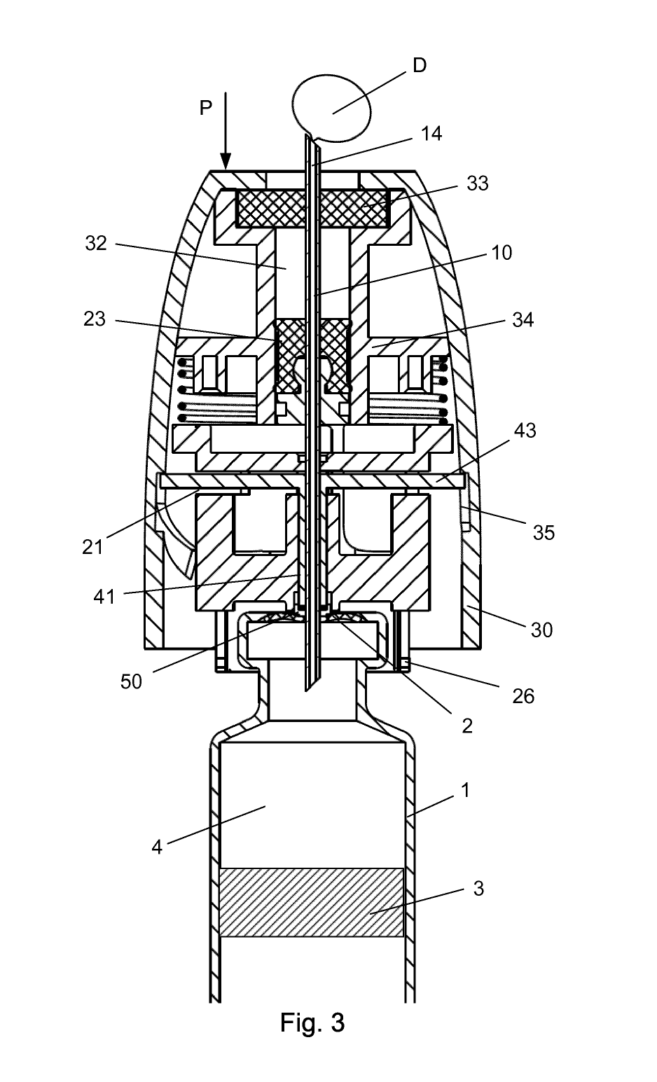

Once the one end 44a of the perpendicular bar 43 is out of the helical part 36 of the track 35 it enters the axial part 37 of the track 35 thus allowing the tip 12 of the needle cannula 10 to reach its fully extended position as depicted in FIG. 3. In this position the injection is finalized.

After the liquid drug has flown into the body of the user, the telescopic shield 30 is removed from the skin of user where after the spring 31 moves the shield 30 back into its initial position of FIG. 1. During this return, the telescopic shield 30 move axially in the distal direction and the motion transforming element 40 rotate in the opposite direction.

The axial hollow part 41 of the axial movement 40 has a wave-shaped proximal surface 45. In the disclosed embodiment, the shape consists of 4 tops and 4 valleys, however any number can be provided.

Between the flexible septum 2 of the cartridge 1 and the motion transforming element 40, a pressure element 50 is provided. This pressure element 50 has a number of guiding elements 51 which are axially guided in similar guiding tracks 22 provided in the hub 20. Further, the pressure element 50 has a proximal end 52 and a wave-shaped distal end 53. This wave-shaped distal end 53 abuts the proximal wave-shaped end 45 of the motion transforming element 40. The wave-shaped surfaces 45, 53 are identical in shape such that when one surface is rotated and the opposite is prevented from rotation one of the surfaces 45, 53 will be forced to move axially.

In the disclosed embodiment, the motion transforming element 40 rotates when the telescopic shield 30 is moved axially as explained above. Since the two wave-shaped surfaces 45, 53 are forced to rotate relatively to each other the proximal end 52 of the pressure element 50 is forced axially further into its abutment with the distal septum 2 of the cartridge 1. This relative rotation forces the pressure element 50 to move axially as the guiding interface 22, 51 between the hub 20 and the pressure element 50 prevents rotation of the pressure element 50.

In the depicted embodiment each of the two wave-shaped surfaces consists of 4 tops and 4 valleys thus requiring a 45 degrees relative rotation to reach its maximum axially departed extension.

When forcing the distal septum 2 of the cartridge 1 further into the interior 4 of the cartridge 1 the volume of the interior 4 decreases. This will consequently press a small amount of liquid drug equal to the decreased volume out through the lumen 14 of the needle cannula 10. Since the volume of the lumen 14 of the needle cannula 10 is very little only a minor decrease in the volume of the interior 4 of the cartridge 1 is needed to actually make a drop of the liquid drug appear on the tip 12 of the needle cannula 10. The expelling of liquid drug is illustrated by the drop “D” in FIGS. 2 and 3 growing in size as the pressure element 50 is pressed further in the proximal direction. The drop “D” thus appearing and growing at the tip 12 of needle cannula 10 as the shield 30 move proximally and the tip 12 penetrates the skin of the user wets the tip 12 of the needle cannula and makes the penetration of the skin less painful. At the same time it minimizes bacteria transportation into the skin and it helps to clean the cutting passage for the tip 12 of the needle cannula 10 as it penetrates the distal septum 33.

Some preferred embodiments have been shown in the foregoing, but it should be stressed that the invention is not limited to these, but may be embodied in other ways within the subject matter defined in the following claims.

1. An injection apparatus comprising:
A cartridge having a distal end sealed by a pierceable flexible septum, and an opposite proximal end sealed by a movable plunger, wherein, the cartridge, the flexible septum and the movable plunger together defines an interior containing a liquid drug,
A needle cannula defining a hollow lumen, which needle cannula is attached to a hub means, and further comprises a distal part and a proximal part which proximal part penetrates the flexible septum such that liquid drug can flow from the interior of the cartridge and through the lumen of the needle cannula upon movement of the movable plunger,
A telescopic shield axially movable relatively to the hub means and urged in a distal direction by a spring,
A motion transforming means which engages the telescopic shield such that axial movement of the telescopic shield is transformed to a rotational movement of the motion transforming means,
An axially movable pressure element engaging the motion transforming means and which pressure element is configured to perform an axial movement relatively to the cartridge upon rotational movement of the motion transforming means, and wherein
telescopic movement of the telescopic shield in a proximal direction rotates the motion transforming means which in turn operates the pressure element to move axially in a proximal direction and wherein the pressure element abuts the flexible septum which is forced proximally upon axial movement of the pressure element such that the volume of the interior of the cartridge decreases.

2. An injection apparatus according to claim 1, wherein the telescopic shield carries a cleaning reservoir.

3. An injection apparatus according to claim 1, wherein the hub structure forms part of an injection device.

4. An injection apparatus according to claim 1, wherein the hub structure forms part of a needle assembly attachable to an injection device.

5. An injection apparatus according to claim 1, wherein the telescopic shield comprises an internal track having a helical part.

6. An injection apparatus according to claim 5, wherein motion transforming structure is guided in the internal track of the telescopic shield.

7. An injection apparatus according to claim 6, wherein the motion transforming structure comprises an axial extending hollow part surrounding the needle cannula and a perpendicular bar having ends.

8. An injection apparatus according to claim 7, wherein at least one end of the perpendicular bar engages the helical part of the internal track of the telescopic shield and wherein the motion transforming structure is axially secured such that at least part of the motion transforming structure perform a rotational movement around its own centre axis upon axial movement of the telescopic shield.

9. An injection apparatus according to claim 7, wherein a proximal end of the hollow part of the motion transforming structure has a wave-shaped surface.

10. An injection apparatus according to claim 9, wherein a distal end surface of the pressure element is wave-shaped and abuts the proximal wave-shaped end of the hollow part of the motion transforming structure.

11. An injection apparatus according to claim 10, wherein the pressure element is axial guided and the wave-shaped surfaces are configured such that rotation of the motion transforming structure forces the pressure element to move axially.

12. An injection apparatus according to claim 11, wherein the pressure element is axially guided in an axial guiding track provided in the hub structure and the motion transforming structure is axially secured in the hub structure.

13. An injection apparatus according to claim 12, wherein a proximal end of the pressure element abuts the flexible septum.

14. An injection apparatus according to claim 13, wherein rotation of the motion transforming structure forces the pressure element further into abutment with the flexible septum such that the volume of the interior of the cartridge decreases.
